(19) United States
(12) Patent Application Publication
(10) Pub. No.: US 2024/0108304 A1
(43) Pub. Date: Apr. 4, 2024

(54) Stethoscope

(71) Applicant: Chukwuemeka Anderson Obi, Charleston, SC (US)

(72) Inventor: Chukwuemeka Anderson Obi, Charleston, SC (US)

(21) Appl. No.: 17/958,526

(22) Filed: Oct. 3, 2022

(51) Int. Cl. A61B 7/04; A61B 5/00; A61B 5/318

(52) U.S. Cl. CPC A61B 7/04; A61B 5/0006; A61B 5/318; A61B 5/7445; A61B 5/7465; A61B 2562/0219; H04B 1/38

(57) ABSTRACT

The present invention relates to a novel form of a remote stethoscope or stethoscope of the metaverse, telemedicine, or telehealth environment which provides advancements including remote operation, use of machine learning and artificial intelligence for assessment and diagnosis of medical conditions, and associated application for transfer, retrieval, and storage of recorded data. The stethoscope data aids in analysis which can correlate with or integrate into an electrocardiogram to help with diagnosing, qualifying, or assessing the severity of diseases. The readings from the stethoscope can be synchronized to mobile devices, smartwatches, and other wearables via Bluetooth. The assembly is high quality, easy to use, and sustainable in nature.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This application generally relates to medical devices. In particular, the application relates to wireless stethoscopes with various advancements designed for virtual environments including telemedicine/telehealth, virtual reality and metaverse environments.

### Description of the Related Art

In the clinic, the stethoscope has been used for over 200 years and it is still an essential tool. Its main function is to test the phonocardiogram (PCG) and heart rate.

### Stethoscope History

The stethoscope was invented in France in 1816 by René Laennec at the Necker-Enfants Malades Hospital in Paris. It consisted of a wooden tube and was monaural. Laennec invented the stethoscope because he was uncomfortable placing his ear on women's chests to hear heart sounds. His device was similar to the common ear trumpet, a historical form of hearing aid; indeed, his invention was almost indistinguishable in structure and function from the trumpet, which was commonly called a “microphone”. Laennec called his device the “stethoscope” (stethoscope, “chest scope”).

The first flexible stethoscope of any sort may have been a binaural instrument with articulated joints not very clearly described in 1829. In 1840, Golding Bird described a stethoscope he had been using with a flexible tube. Bird was the first to publish a description of such a stethoscope but he noted in his paper the prior existence of an earlier design (which he thought was of little utility), which he described as the snake ear trumpet. Bird's stethoscope had a single earpiece. In 1851, Irish physician Arthur Leared invented a binaural stethoscope, and in 1852 George Philip Cammann perfected the design of the stethoscope instrument (that used both ears) for commercial production, which has become the standard ever since. Cammann also wrote a major treatise on diagnosis by auscultation, which the refined binaural stethoscope made possible. By 1873, there were descriptions of a differential stethoscope that could connect to slightly different locations to create a slight stereo effect, though this did not become a standard tool in clinical practice. The medical historian Jacalyn Duffin has argued that the invention of the stethoscope marked a major step in the redefinition of disease from being a bundle of symptoms, to the current sense of a disease as a problem with an anatomical system even if there are no noticeable symptoms. This re-conceptualization occurred in part, Duffin argues, because prior to the stethoscopes, there were no non-lethal instruments for exploring internal anatomy. Rappaport and Sprague designed a new stethoscope in the 1940s, which became the standard by which other stethoscopes are measured, consisting of two sides, one of which is used for the respiratory system, the other for the cardiovascular system. The Rappaport-Sprague stethoscope was later made by Hewlett-Packard. HP's medical products division was spun off as part of Agilent Technologies, Inc., where it became Agilent Healthcare. Agilent Healthcare was purchased by Philips which became Philips Medical Systems, before the walnut-boxed, original Rappaport-Sprague stethoscope was finally abandoned in 2004, along with Philips' brand (manufactured by Andromed, of Montreal, Canada) electronic stethoscope model. The Rappaport-Sprague model stethoscope was heavy and short (18-24 in [46-61 cm]) with an antiquated appearance recognizable by its two large independent latex rubber tubes connecting an exposed-leaf-spring-joined-pair of opposing “f”-shaped chrome-plated brass binaural ear tubes with a dual-head chestpiece.

Several other minor refinements were made to stethoscopes, until in the early 1960s David Littmann, a Harvard Medical School professor, created a new stethoscope that was lighter than previous models and had improved acoustics. In the late 1970s, 3M-Littmann introduced the tunable diaphragm: a very hard (G-10) glass-epoxy resin diaphragm member with an overmolded silicone flexible acoustic surround which permitted increased excursion of the diaphragm member in a “z”-axis with respect to the plane of the sound collecting area. The left shift to a lower resonant frequency increases the volume of some low frequency sounds due to the longer waves propagated by the increased excursion of the hard diaphragm member suspended in the concentric acoustic surround. Conversely, restricting excursion of the diaphragm by pressing the stethoscope diaphragm surface firmly against the anatomical area overlying the physiological sounds of interest, the acoustic surround could also be used to dampen excursion of the diaphragm in response to “z”-axis pressure against a concentric fret. This raises the frequency bias by shortening the wavelength to auscultate a higher range of physiological sounds.

In 1999, Richard Deslauriers patented the first external noise reducing stethoscope, the DRG Puretone. It featured two parallel lumens containing two steel coils which dissipated infiltrating noise as inaudible heat energy. The steel coil “insulation” added 0.30 lb to each stethoscope. In 2005, DRG's diagnostics division was acquired by TRIMLINE Medical Products. In 2015, Dr. Tarek Loubani announced an open-source 3D-printed stethoscope based on the 1960s-era Littmann Cardiology 3 stethoscope. The 3D-printed equivalent is nearly an order of magnitude more affordable than the aforementioned non-3D-printed stethoscope and is intended to make the medical device more accessible to obtain, particularly in developing countries.

With the advent of the modern electronic stethoscope, a visual indication of heart sounds or chest sounds can be made which aids in diagnosis. Electronic stethoscopes allow the physician to discern subtle differences in the frequency, intensity and duration of many sounds. However, it is still incumbent on the physician to correctly analyze the recorded and displayed sounds and diagnose the underlying abnormalities therefrom.

Multiple advancements regarding stethoscopes are seen by looking at prior art. For instance, an electronic stethoscope system bearing US patent 2,009,0316925A1 is issued to Leonard Eisenfeld, Jonathan M. Hill, and Ronald S. Adrezin. The patent relates to an electronic stethoscope head that has a head member with a contact surface for contact with a patient's body, a transducer in the head member, and an adhesive on the contact surface. A processing system for an electronic stethoscope includes a conditioning circuit configured to receive a transducer signal from a transducer and to be capable of amplifying and/or filtering the transducer signal, to yield a conditioned signal. There is also a signal processor system configured to subject the conditioned signal to an audio editing process. Bodily sounds are detected by applying an electronic stethoscope head a patient's body, generating a patient sonograph of the patient's bodily sounds, and comparing the patient sonograph to a reference sonograph. An electronic stethoscope system may include an accessory device and control circuitry to control the accessory device when abnormal bowel sounds are detected or no bowel sounds are detected for a predetermined interval.

A U.S. Pat. No. 9,445,779B2 on Infrasonic stethoscope for monitoring physiological processes is issued to National Aeronautics and Space Administration (NASA). The patent discusses an infrasonic stethoscope for monitoring physiological processes of a patient, which includes a microphone capable of detecting acoustic signals in the audible frequency bandwidth and in the infrasonic bandwidth (0.03 to 1000 Hertz), a body coupler attached to the body at a first opening in the microphone, a flexible tube attached to the body at a second opening in the microphone, and an earpiece attached to the flexible tube. The body coupler is capable of engagement with a patient to transmit sounds from the person, to the microphone and then to the earpiece.

A US patent 2,022,0096043A1 discloses wireless stethoscope and method of use thereof is issued to Pulmonary APPS LLC. The patent relates to a wireless stethoscope that is described as having wireless sensors that are enclosed in disposable pads so that the same pads are not used on more than one patient, preventing cross-infection of patients associated with conventional stethoscopes. The present wireless stethoscope also detects pulmonary sounds and cardiac sounds, allowing the user to monitor one or the other without interference. Also described is a method for diagnosing a pulmonary condition using the wireless stethoscope.

A US patent 2,008,0013747A1 Digital stethoscope and monitoring instrument is issued to Bao Tran. The patent discusses an electronic stethoscope which includes a microphone; an accelerometer to detect stethoscope movement, a processor coupled to the microphone and the accelerometer; and a speaker coupled to the processor to reproduce a biological sound.

A U.S. Pat. No. 5,010,889A on intelligent stethoscope is issued to Mark S. Bredesen and Elliott D. Schmerler. The patent relates to an intelligent stethoscope for performing auscultation and for automatically diagnosing abnormalities using body sounds in which body sounds are received, digitized and stored in memory. The body sounds are recorded from a plurality of locations on the body, and all of the sounds are categorized according to specific characteristics to form a matrix of information. The generated matrix is then compared against a plurality of stored matrices using a technique similar to signature analysis. Each of the stored matrices contain information indicative of known abnormalities such as specific heart murmurs and lung abnormalities. When a matrix match is found, the diagnosis is displayed on an LCD display formed in the body of the stethoscope. The LCD display is also capable of displaying a visual representation of the recorded body sounds. The present invention is applicable to heart sounds, lung sounds, and sounds from blood vessels. A wide variety of heart and lung abnormalities along with their signatures are described, and the specific steps required for the signature analysis is described.

A U.S. Pat. No. 8,827,920B2 on elemedical stethoscope is issued to SNU R&DB Foundation. The patent discloses a telemedical stethoscope, which automatically diagnoses a disease, records visually and auditorily, and stores the stethoscope data on a screen. The present invention enhances primary diagnosis and treatment effect for a patient by transmitting/receiving the data to/from a doctor at a medical center and by receiving a telemedicine service. In addition, the present invention transmits the data to a health management program so as to be used for healthcare decisions of a patient.

A portable watch-type stethoscope in Korean Utility model Registration No. 0131518 and U.S. Pat. No. 5,737,429 embeds a microphone in a sound collecting tube of a sound collecting plate to convert an auscultation sound in an inner body into an electrical signal and to cause a doctor to listen to and watch a body sound with a speaker and display.

A mobile phone with a stethoscope in Korean Utility model Registration No. 0389343 and PCT Patent Application No. PCT/KR2005/003800 has advantageous effects that transmit and receive auscultation auscultated thereby through transceiver function in the mobile phone to a remote location to receive a telemedicine service.

The inherent disadvantage of the aforementioned prior art is that these devices provide various features, however, their ease-of-use for patients at home is questionable. Using them without prior training and knowledge is a difficult task. Therefore, despite the availability of numerous providing functionalities in their own domain, there is still a need for a device that provides the consistent necessary functionality with ease of use and enhanced advantages.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease-of-use ability of these assemblies is also questionable. The current invention proposes an advancement in stethoscope assembly with various modern features including, but not limited to, the ability to remotely listen to sounds from human and animal body, the use of machine learning and artificial intelligence to analyze and interpret sounds from humans and animals, remote examination, and the maintenance of social distancing to prevent the need to touch a human's body.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The present invention seeks to improve upon prior stethoscope assemblies and provide an enhanced system to perform the designated functionality.

Another object of the present invention is to provide a stethoscope that uses a microphone to replace the hearing function of human ears.

It is a further objective of the present invention to provide an improved structure made of high-quality materials including but not limited to hard plastic, silicone, rubber, or other synthetic material which maintain their effectiveness over a longer period of time.

It is also an objective of the invention to use machine learning and artificial intelligence to analyze and interpret sounds from humans and animals.

It is another objective of the present invention to provide stethoscope data analysis functionality that can correlate with or integrate with an electrocardiogram (ECG) to help with diagnosing, qualifying, or assessing the severity of the disease.

It is further the objective of the invention to provide assembly whose readings from the stethoscope can be synchronized to mobile devices, smartwatches, and other wearables. The device will integrate with smart devices via Bluetooth.

It is also the objective of the invention to provide a device where sound will be analyzed with machine learning and artificial intelligence resulting in the interpretation of the sound in humans and animals.

It is another object of the present invention to provide a stethoscope that may audio-visually record and store a result diagnosed per the auscultation mode and remotely transmit to provide a telemedicine service to a patient.

It is further the object of the present invention to provide a digital or smart stethoscope that is universal in nature and can be used in virtual reality (VR), augmented reality (AR), and the metaverse environment.

Thus, it is the objective to provide a new and improved stethoscope device with an associated application that can be integrated into web applications and mobile applications to allow for remote examination of humans and animals for diagnostic purposes.

This Summary is provided merely for the purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWING

non-limiting and non-exhaustive embodiments of the present invention are described with reference to the following drawings. The system and method of the present invention will now be described with reference to the accompanying flow chart drawing figure, in which:

FIG. 1 is a top, front, perspective view of the Stethoscope as per preferred embodiments of the invention;

FIG. 2 is a top, right-side, perspective view of the Stethoscope as per preferred embodiments of the invention;

FIG. 3 is a top, back, perspective view of the Stethoscope as per preferred embodiments of the invention;

FIG. 4 is a top, left-side, perspective view of the Stethoscope as per preferred embodiments of the invention;

FIG. 5 is a back, left-side, perspective view of the Stethoscope as per preferred embodiments of the invention;

FIG. 6 is a front, left-side, perspective view of the Stethoscope as per preferred embodiments of the invention; and,

FIG. 7 is a back perspective view of the Stethoscope as per preferred embodiments of the invention.

Skilled artisans will appreciate that elements in the figures are illustrated for simplicity and clarity and have not necessarily been drawn to scale. For example, the dimensions of some of the features in the figures may be exaggerated relative to other elements to improve understanding of embodiments of the present invention. The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In one aspect of the present invention, the current invention discloses an advanced stethoscope to determine disease, store, analyze and transmit data to a cloud-based interface.

The assembly comprises a stethoscope case, a sound collecting plate, a central control device, a display window, an on/off button, a microphone, and a speaker.

The current assembly as per preferred embodiments provides and causes inner units in the stethoscope (100) to operate the program of the stethoscope mode, and display the data in a display unit of the stethoscope. The assembly is different from the usual structure of a stethoscope, where the sensing element is embedded in the circular region. The upper handle has the display screen (101) while the < > (102) is for selection, menu selection, and setting adjustments like volume/output. The “O” (103) is for the menu, settings and home page. The handle is also used to hold the instrument with ease.

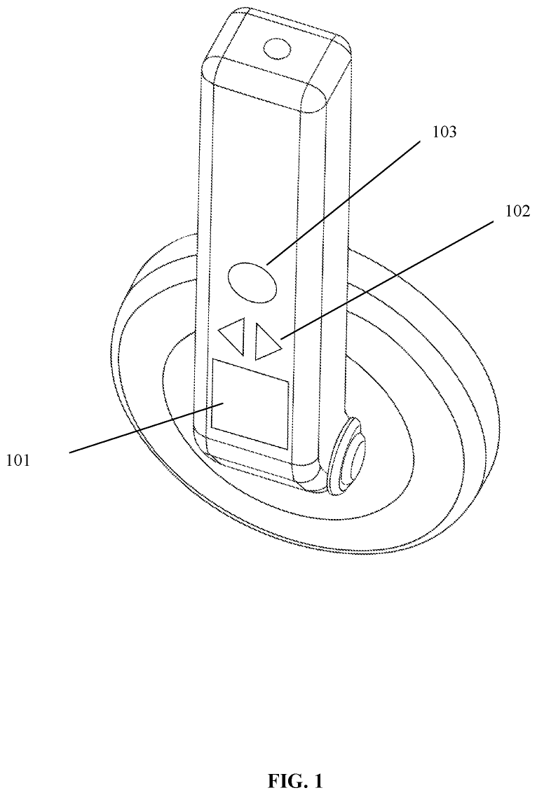

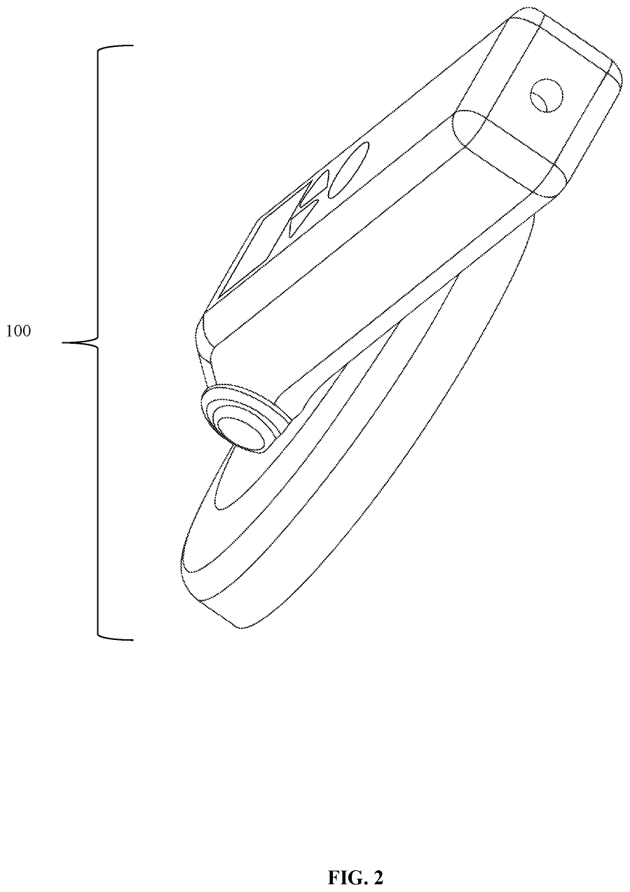

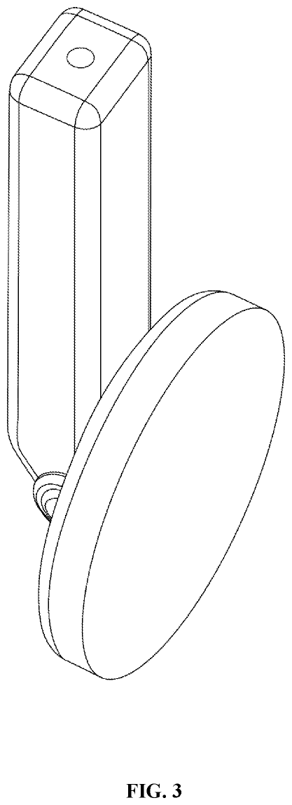

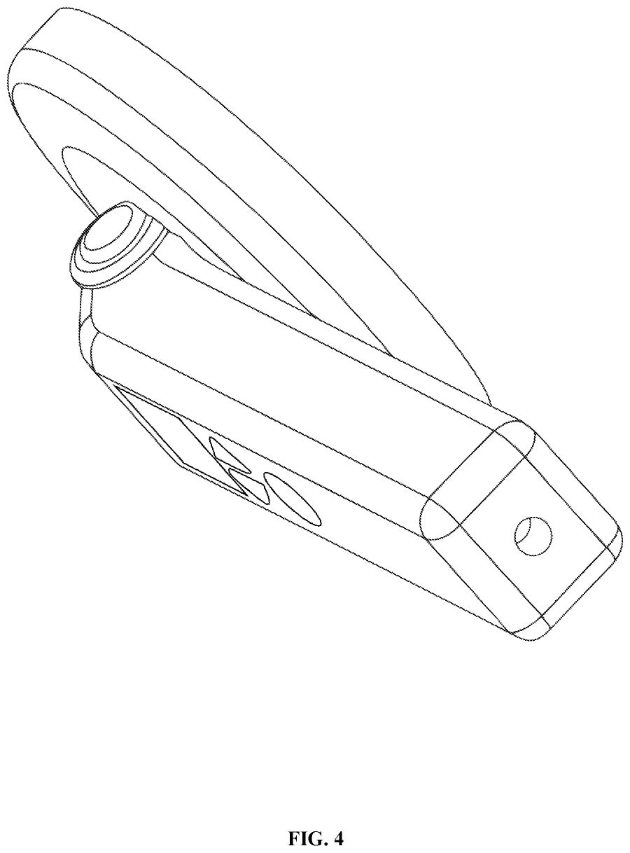

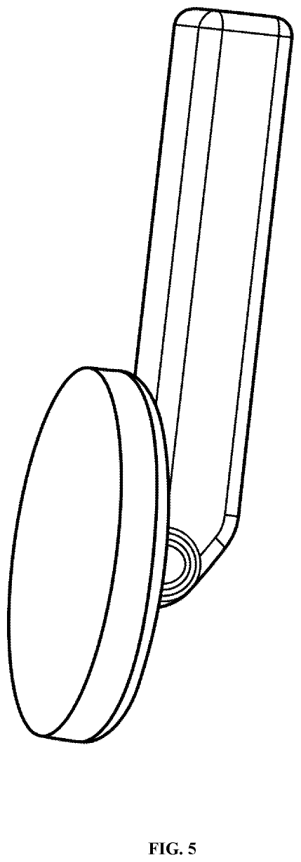

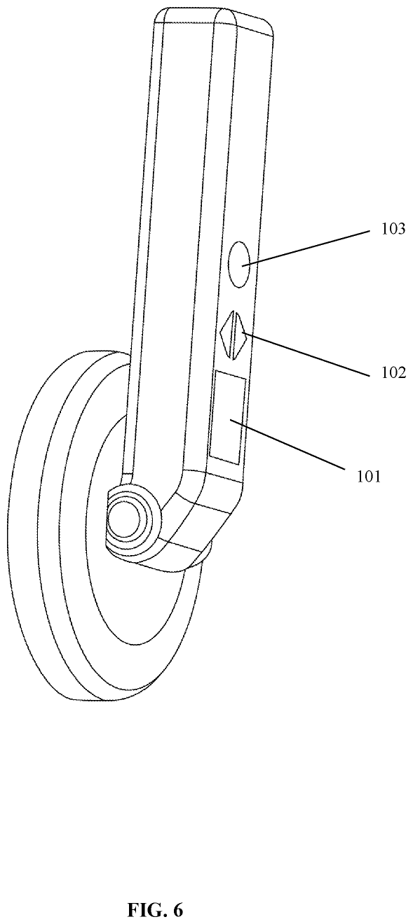

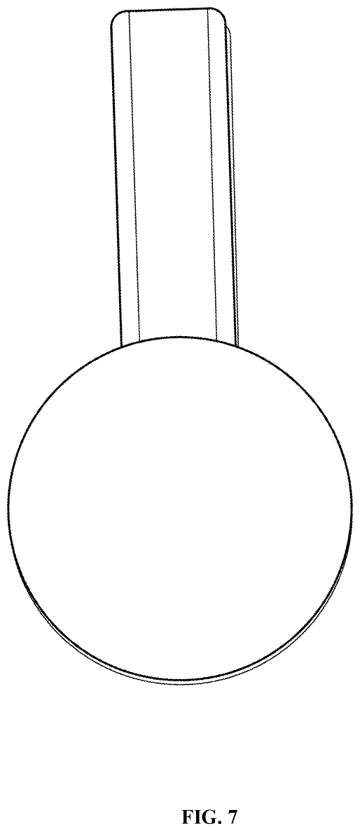

As illustrated in FIG. 1-FIG. 7, the stethoscope assembly's central control device and power supply are built in the inner case which is internally equipped with one or more sound-collecting microphones. The LCD (Liquid Crystal Display) is built on the case, with a display of battery life, time, date, and medical data readings. The < > (102) button for selection, menu selection, and setting adjustments like volume/output. The “O” (103) button for menu, settings, and home page.

The current assembly as per preferred embodiments has the ability to remotely listen to sounds from human and animal body including heart, lung, abdomen and blood vessels to help in the care of the patient. The assembly is able to listen to the heart region, pulmonary region, a neck carotid artery region and a bowel region in the digestive system and the data is stored in the central control unit.

The stethoscope (100) of the present invention operates like the following. When a patient is primarily checked, the first auscultation region is a heart region, a second region is a pulmonary region, a third region is a neck region and a fourth region is a bowel region. However, the auscultation order in the auscultation mode may be situationally changed.

The current assembly as per preferred embodiments uses machine learning and artificial intelligence to analyze and interpret sounds from humans and animals. The structure of the stethoscope also provides associated applications, which can be integrated to web applications and mobile applications to allow for remote examination of humans and animals for diagnostic purposes. The present invention may store diagnosis data and transmit the data through an associated application to its designated server. The data can be used by a doctor in a medical center or can be shared with any caretaker. The present invention may transmit the data to an attached device linked with a health management program to be used for healthcare.

The data collected is transmitted to an attached device linked with a health management program and used for disease management and personal health management. The stethoscope of the present invention can be used in healthcare. The smart stethoscope can be used in telemedicine, hospitals and various clinical settings, and is especially helpful to patients and providers during times of social distancing in

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. An enhanced structure of artificial intelligence-assisted stethoscope comprising:
a central control device and a power supply are built in a case that is integrally equipped with a sound collecting plate embedding a microphone, wherein an LCD (Liquid Crystal Display) is built on the case, wherein button < > (102) is for selection, menu selection and setting adjustment like volume/output, wherein the “O” (103) button is for menu, settings and home page; and,
a software module receiving data from the sensors to detect changes in patient sounds.

2. The system of claim 1, comprising an accelerometer to perform necessary readings.

3. The system of claim 1, comprising a sound transducer coupled to the wireless transceiver to communicate audio through the network.

4. The system of claim 1, wherein the sensor monitors the patient's lungs, heart, and other sounds inside the body.

5. The system of claim 1, wherein the screen mounted on the stethoscope assembly displays data displayed in a numerical format with recommended values.

6. The system of claim 1, wherein the noted values are transferred to smart devices through Bluetooth connectivity.

7. The system of claim 1, wherein the Stethoscope data can correlate with or integrate into an electrocardiogram (EKG) to help with diagnosing, qualifying or assessing the severity of the disease.

8. The system of claim 1, wherein specifically designed for digital environments such as virtual reality (VR), augmented reality (AR), and the metaverse environment.

9. The system of claim 1, wherein the Stethoscope serves as a remote stethoscope specifically for telemedicine/telehealth.
